(19) United States
(12) Patent Application Publication
(10) Pub. No.: US 2026/0248630 A1
(43) Pub. Date: Aug. 27, 2026

(54) TREATMENT SYSTEMS AND METHODS FOR A DISEASED AORTA

(71) Applicant: W. L. Gore & Associates, Inc., Newark, DE (US)

(72) Inventor: Cathryn Jensen, Flagstaff, AZ (US)

(21) Appl. No.: 19/544,547

(22) Filed: Feb. 19, 2026

Related U.S. Application Data
(60) Provisional application No. 63/761,676, filed on Feb. 21, 2025.

(51) Int. Cl. A61F 2/966; A61F 2/07; A61F 2/95

(52) U.S. Cl. CPC A61F 2/966; A61F 2/07; A61F 2002/9534

(57) ABSTRACT

Devices and methods to treat a diseased portion of an aorta. A method of implanting an endoprosthesis within the aorta may include positioning the endoprosthesis within the aorta in a collapsed state with a constraint and deploying the endoprosthesis to the deployed diameter. The aorta has a true lumen and a false lumen due to the aorta having a defect, the true lumen defining a true lumen diameter and the false lumen formed by a false lumen wall defining a false lumen diameter. The deployed diameter of the endoprosthesis is such that the endoprosthesis extends along the true lumen and along at least a portion of the false lumen at a position where the defect is located in the aorta such that the endoprosthesis defines a flow bypass through a diseased portion of the aorta.

### CROSS-REFERENCE TO RELATED APPLICATIONS

This application claims the benefit of Provisional Application No. 63/761,676, filed Feb. 21, 2025, which is incorporated herein by reference in its entireties for all purposes.

### BACKGROUND

An aortic dissection is initiated by a tear in the aortic intima which may extend proximally or distally from the location of the initial injury as the intimal and adventitial layers of the aortic wall separate, allowing blood to flow between the layers to form a false lumen. Common complications are false lumen rupture and obstruction of blood flow into branch vessels.

Most often, the primary intimal tear of a de novo dissection originates in the ascending aorta (a Type A dissection), often within the first 3 cm above the aortic root. Historical clinical experience suggests an elevated risk of immediate mortality from untreated de novo Type A dissection. An acute Type A aortic dissection is therefore a medical emergency requiring prompt intervention to repair the ascending aorta and avoid a catastrophic rupture. Regarding chronicity, American College of Cardiology (ACC) and American Heart Association (AHA) further define four temporal types of aortic dissection including hyperacute aortic dissection defined at 24 hours or less after system onset, acute aortic dissection defined at 1-14 days after system onset, subacute aortic dissection at 15-90 days after system onset, and chronic aortic dissection at greater than 90 days after system onset.

Open surgery is the standard Type A treatment. During open surgical repair, the surgeon resects and replaces the ascending aorta. During repair, the surgeon may use techniques to address other risks presented by proximity of the dissection to the heart, including coronary malperfusion, cardiac tamponade, and/or acute aortic insufficiency which may lead to interventions on the aortic valve and/or coronary arteries in addition to aortic repair.

### SUMMARY

Despite the risk of Type A dissection, some patients are not offered open surgical repair due to patient-specific, including but not limited to age or BMI, or other disease-related risk factors. Because open surgery is the standard Type A treatment, endovascular treatment is used as an option for patients at high risk for open repair. In an endovascular repair, the surgeon implants an endovascular device to exclude blood flow through the primary entry tear, depressurize the false lumen, and restore flow to the “true” aortic lumen. In contrast to open surgery, endovascular treatment of Type A dissections does not require median sternotomy, cardiopulmonary bypass (CPB), or adjunctive hypothermic arrest.

In some embodiments as discussed herein, an endoprosthesis is endoluminally delivered through the true lumen to bypass the false lumen of the aorta of the patient to treat the aortic dissection. The endoprosthesis may cover at least the primary entry tear, which can help to stabilize the aorta and reduce risk of rupture. In some embodiments, bypassing blood flow to the false lumen can cause the false lumen to thrombose. The endoprosthesis may have geometric features selected based on a maximum transverse diameter of the sum of a true lumen of the aorta and the false lumen, sometimes referred to as total aorta diameter.

According to one example (“Example 1”), a method of implanting an endoprosthesis within an aorta of a patient includes positioning the endoprosthesis within the aorta in a collapsed state with a constraint, the aorta having a true lumen and a false lumen due to the aorta having a defect, the true lumen defining a true lumen diameter, the endoprosthesis having a deployed diameter and a collapsed diameter and deploying the endoprosthesis to the deployed diameter such that the endoprosthesis extends along the true lumen and along at least a portion of the false lumen at a position where the defect is located in the aorta such that the endoprosthesis defines a flow bypass through a diseased portion of the aorta.

Optionally, the false lumen diameter is greater than the true lumen diameter.

According to another example (“Example 2”), further to Example 1, the constraint includes a first constraint and a second constraint and deploying the endoprosthesis includes releasing the first constraint constraining the endoprosthesis such that the endoprosthesis expands from the collapsed diameter to an intermediate diameter that is greater than the collapsed diameter, repositioning the endoprosthesis, and releasing the second constraint constraining the endoprosthesis such that the endoprosthesis expands from the intermediate diameter to the deployed diameter.

According to another example (“Example 3”), further to either Example 1 or 2, the method includes pulling back a portion of the constraint such that left and right coronary arteries are left substantially unobstructed by the endoprosthesis.

According to another example (“Example 4”), further to any Examples 1-3, the endoprosthesis is implanted within the ascending aorta.

According to another example (“Example 5”), further to any Examples 1-4, the endoprosthesis diameter is about 31 mm, 34 mm, 37 mm, 40 mm, 45 mm, 49 mm, 53 mm or more.

According to another example (“Example 6”), further to any Examples 1-5, the method includes receiving information regarding a false lumen diameter and selecting the deployed diameter of the endoprosthesis to be at least as large as the true lumen diameter.

According to another example (“Example 7”), further to any Examples 1-6, the diseased portion of the aorta is formed by an aortic dissection.

According to another example (“Example 8”), further to any Examples 1-7, the deployed diameter of the endoprosthesis is selected to be from 6% to 33% or optionally from 20% to 30% larger than a total aorta diameter defined by a sum of the false lumen diameter and the true lumen diameter.

According to another example (“Example 9”), further to any Examples 1-8, the deployed diameter is selected based on overall diameter measurements of the aorta at a proximal aortic neck, a distal aortic neck, and a maximum transverse aortic diameter.

According to another example, (“Example 10”), further to any Examples 1-9, the deployed diameter is selected to be from 6% to 33% larger than a diameter of the aorta proximal to a tear of the diseased portion.

According to another example, (“Example 11”), further to any Examples 1-10, the endoprosthesis extends within the true lumen and the portion of the false lumen.

According to one example (“Example 12”), a method of assessing an ascending aorta of a patient includes measuring at least one measurement of a transverse aortic diameter that is a sum of a diameter of a true lumen and diameter of a false lumen of the ascending aorta, selecting a deployed diameter of an endoluminal device to be compatible with any of the measured transverse aortic diameters, and implanting the endoluminal device within the aorta.

According to another example (“Example 13”), further to Example 12, the method includes ensuring the distance between a distal coronary artery and the proximal extent of a defect associated with the false lumen is about 2 cm or longer. Optionally, the defect is a proximal intimal tear in the aorta.

According to another example (“Example 14”), further to either Examples 12 or 13, the method includes at least one of measuring a distance between a coronary artery and a proximal extent of a defect, measuring a distance between a distal extent of the defect and a brachiocephalic artery, measuring a diameter of a proximal aortic neck, measuring a diameter of a distal aortic neck, measuring a maximum diameter of the defect; and measuring a total length of the ascending aorta.

According to another example (“Example 15”), further to any of Examples 12-14, the method further includes selecting the endoluminal device with the deployed diameter being oversized relative to the transverse aortic diameter.

According to one example (“Example 16”), an implantable system configured to be implanted within an aorta of a patient includes an implantable medical device expandable from a collapsed diameter to a deployed diameter, wherein the deployed diameter is sized to a total diameter of the aorta including a true lumen and a false lumen, a first constraint configured to maintain the implantable medical device at the collapsed diameter, a second constraint configured to maintain the implantable medical device at an intermediate diameter between the collapsed diameter and the deployed diameter.

According to another example (“Example 17”), further to Example 16, the total of the diameter of the aorta is a diameter of aorta positioned within a proximal aortic neck of the aorta.

According to another example (“Example 18”), further to any of Examples 16 or 17, a diameter of the implantable medical device is oversized relative to the total diameter of the aorta.

According to another example (“Example 19”), further to any one of Examples 16-18, a diameter of the implantable device is between about 27 and about 53 mm.

According to another example (“Example 20”), further to any one of Examples 16-19, the implantable medical device includes a stent component and a graft component.

The foregoing Examples are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description, serve to explain the principles of the disclosure.

FIG. 1 is a representation of a human anatomy showing an aorta, in accordance with an embodiment;

FIGS. 2A-2C are representations of the aorta of FIG. 1 including a diseased portion, in accordance with an embodiment;

FIG. 2D is a representation of the aorta of FIG. 1 showing the Stanford Classification for an aortic dissection;

FIG. 3 shows a delivery system including at least one constraint configured to constrain an endoprosthesis, according to some embodiments;

FIG. 4 shows the delivery system of FIG. 3 including at least two constraints configured to constrain the endoprosthesis, according to some embodiments;

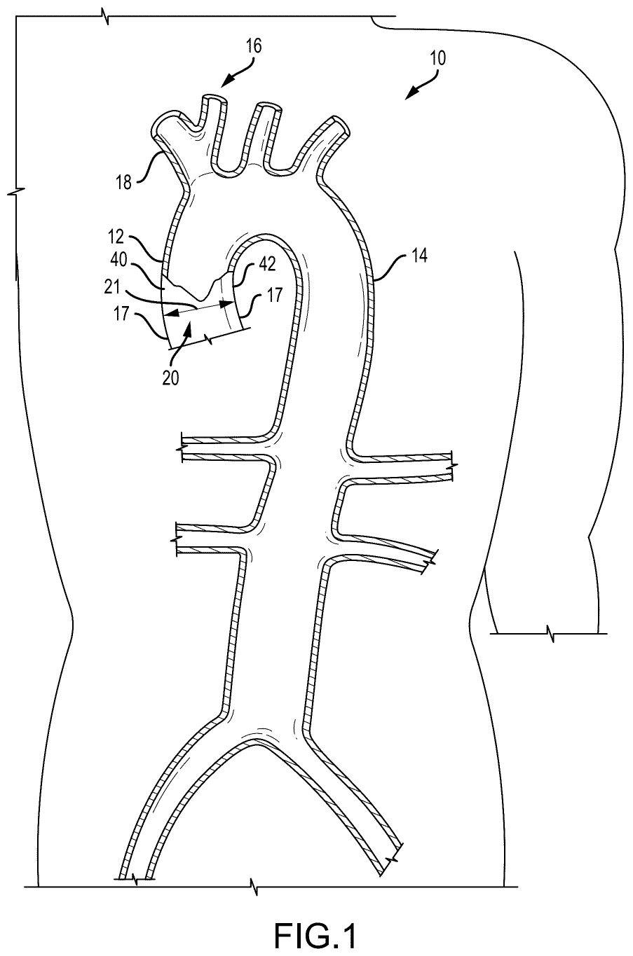

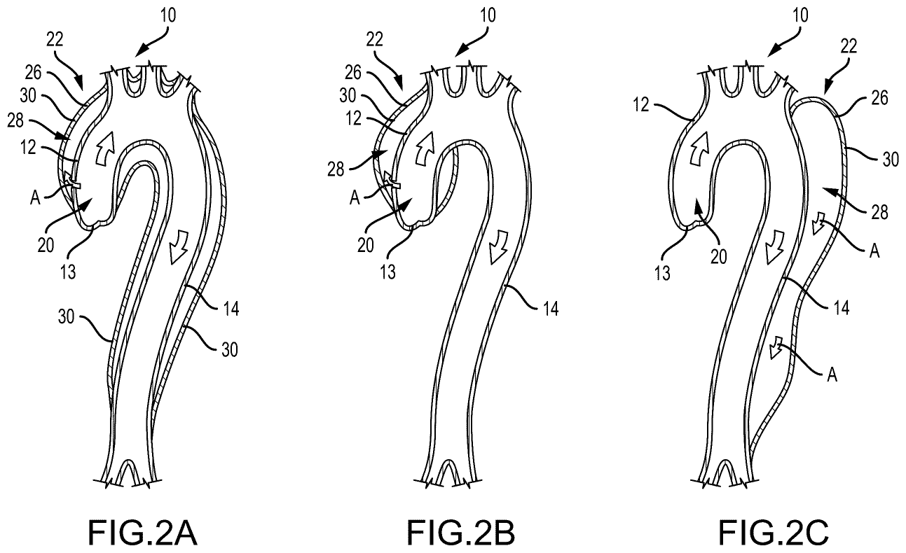

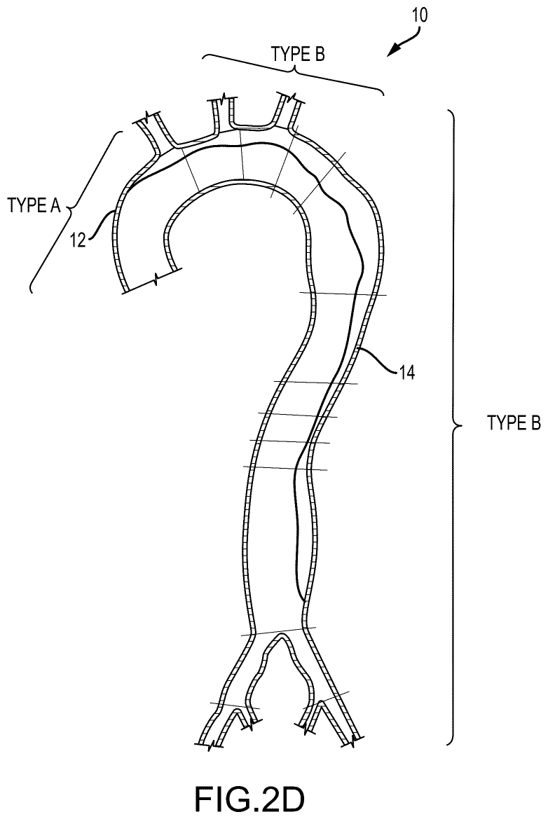

FIG. 2D is a representation of the aorta of FIG. 1 showing the Stanford Classification for an aortic dissection;

FIGS. 5A-5B are representations of a first assessment protocol for an ascending aorta, in accordance with an embodiment;

FIGS. 6A-6B are representations of another assessment protocol for an ascending aorta, in accordance with an embodiment;

FIGS. 7A-7B are representations of a further assessment protocol for an ascending aorta, in accordance with an embodiment;

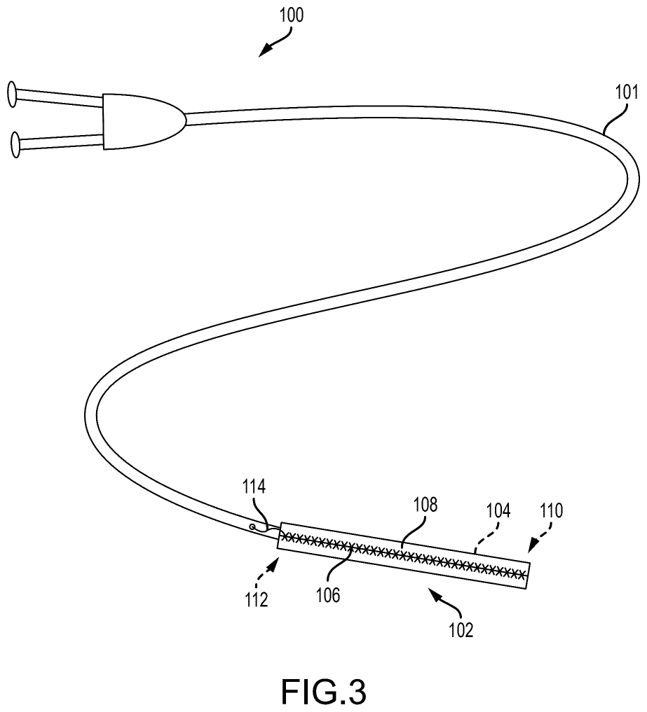

FIG. 8 shows the delivery system of FIG. 3 including a housing, in accordance with an embodiment;

FIGS. 9A-9B shows the delivery system delivering the endoprosthesis to the aorta, in accordance with an embodiment;

FIG. 10 shows a deployment of the endoprosthesis to an intermediate diameter, in accordance with an embodiment;

FIG. 11 shows optional repositioning of the endoprosthesis at the intermediate diameter, in accordance with an embodiment;

FIG. 12 shows a deployment of the endoprosthesis to a deployed diameter, in accordance with an embodiment;

FIG. 13 shows optional repositioning of the endoprosthesis at the deployed diameter, in accordance with an embodiment;

FIG. 14 shows a constraint with a first deployment line section routing pattern, prior to deployment, in accordance with an embodiment;

FIG. 15 shows the constraint with the first deployment line section routing pattern of FIG. 14, after deployment, in accordance with an embodiment;

FIG. 16 shows the constraint with a second deployment line section routing pattern, in accordance with an embodiment;

FIG. 17 shows a first configuration of the constraint during actuation of the second deployment line section of FIG. 16, in accordance with an embodiment;

FIG. 18 shows a second configuration of the constraint of FIG. 17 during continued actuation of the second deployment line section, in accordance with an embodiment;

FIG. 19 shows a third configuration of the constraint of FIG. 18 during continued actuation of the second deployment line section, in accordance with an embodiment;

FIG. 20 shows another configuration of the constraint according to some embodiments after a deployment line has been removed from the constraint, in accordance with an embodiment;

FIG. 21 shows the endoprosthesis post-deployment within the aorta, in accordance with an embodiment; and

FIG. 22 shows a partial view of the endoprosthesis post-deployment with the constraint pulled back, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Endoluminal devices are frequently used to treat the vasculature of human patients. These treatments or procedures are commonly referred to as intraluminal or endovascular procedures. Such devices often include a constraint such as a sleeve. As used herein, the term “constraint” refers to a primary, secondary, tertiary, etc., sleeve, sheath, or the like, that constrains an endoluminal device toward a collapsed configuration or outer peripheral dimension suitable for endoluminal delivery of the device to a treatment portion of the vasculature of a patient.

For purposes of the disclosure, the term “constrain” may mean (i) to limit the expansion, either through self-expansion or assistance by a device (e.g., a balloon), of the diameter of at least a portion of a medical device or (ii) to cover or surround but not otherwise restrain at least a portion of a medical device (e.g., for storage or biocompatibility reasons and/or to provide protection to the medical device and/or the vasculature). For reference, the term “diameter” is not meant to require a circular cross-section and is instead to be understood broadly to reference a maximum transverse cross-sectional dimension of the medical device.

As used herein, the term “line” such as “deployment line” or “steering line” refers to any type of string, cord, thread, fiber, or wire, and can be comprised of metallic, polymeric, or natural materials, including conventional medical grade materials such as nylon, polyacrylamide, polycarbonate, polyethylene, polyform aldehyde, polymethylmethacrylate, polypropylene, polytetrafluoroethylene, expanded polytetrafluoroethylene, polytrifluorochlorethylene, polyvinylchloride, polyurethane, elastomeric organosilicon polymers; metals such as stainless steels, cobalt-chromium alloys and nitinol; and high strength polymer fibers such as ultra-high molecular weight polyethylene fibers or aramid fibers.

Throughout this specification and the claims, the terms “distal” or “leading” may refer to a relative location on a device which is closer to the end of the device that is inserted into and progressed through the vasculature of a patient. The terms “proximal” or “trailing” may refer to a relative location on a device which is closer to the end of the device that is located outside of the vasculature of a patient. In related terms, the terminology “distal end” can be interpreted as a “far end” and “proximal end” and a “near end.” Similarly, “distal” and “proximal” terminology may be applied to describe portions of the vasculature, such as the aorta.

This disclosure is not meant to be read in a restrictive manner. For example, the terminology used in the application should be read broadly in the context of the meaning those in the field would attribute such terminology.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

Devices, systems, and methods of endoluminally delivering an expandable implantable endoprosthesis in accordance with various embodiments are disclosed herein for treating disease of human vasculature. Although the description below and figures are illustrated in the context of treating an aorta 10, including an ascending aorta 12, it should be appreciated that the present disclosure can be applied to treatment of other portions of the vasculature, including, for example, any disease where a larger vessel and one or more branch vessels are to be treated.

Endoprostheses may include different stages of deployment for implantation in a body of a patient. For example, the endoprosthesis may include an undeployed, delivery configuration in which at least a portion of the endoprosthesis is contained in at least one constraint or sleeve that constrains the endoprosthesis for delivery into the vasculature of a patient. When the endoprosthesis has been delivered into the vasculature of a patient, the constraint is removed, allowing the device to be deployed and expanded in a manner complementary to the vasculature. For reference, the term “removed” as used with respect to the constraint is synonymous with “released,” and does not require physical removal from the body, though in some cases, the constraint may be at least partially removed from the body.

The constraint may be comprised of, for example, expanded polytetrafluoroethylene (ePTFE), polyester, polyurethane, fluoropolymers, such as perfluoroelastomers and the like, polytetrafluoroethylene, silicones, urethanes, polyethylene, such as ultra-high molecular weight polyethylene, expanded polyethylene, and the like, aramid fibers, and combinations thereof. Other instances for the sleeve material may include high strength polymer fibers such as ultra-high molecular weight polyethylene fibers or aramid fibers. The sleeve may include a bioactive agent. Any sleeve which may be used to constrain an endoprosthesis is in accordance with the present disclosure.

Referring to FIG. 1, a representation of an aorta 10 of a patient is shown. The aorta 10 includes an ascending aorta 12, a descending aorta 14, and plurality of branches 16, including a brachiocephalic artery 18. The ascending aorta 12 terminates at a root 13 (FIG. 2A) and includes two coronary arteries 15 (FIG. 5A). The aorta 10 defines a true lumen 20 between walls 17 of the aorta 10, which allows blood flow through the vasculature. The true lumen 20 defines a true lumen diameter 21. The aorta 10 also defines a greater outer curve 40 and an inner curve 42.

As discussed above, and as shown in FIGS. 2A-2D, patients may develop a diseased portion 22 within the aorta 10, including but not limited to, an aortic dissection. Aortic dissection occurs when layers of at least one wall 17 of the aorta 10 separate from each other at a tear region 24 (FIG. 6A), allowing blood to flow through the true lumen 20 and between the layers of the at least one wall 17 of the aortic wall, as illustrated by arrows A. The blood may pool between the layers of the aortic wall, leading to separation of layers of the aortic wall and formation of a defect 26 at a false lumen wall 30, where the defect 26 may include a lesion or a dissection. The defect 26 can be localized along the aorta 10 or may extend proximal or distal to an opening in the intimal wall of the aorta 10. The defect 26 defines a false lumen 28 therein that collects the pooled blood. The false lumen 28 may cause outward swelling of the aorta 10 such that the false lumen wall 30 extends outwardly relative to the wall 17 and increases a total aortic diameter of the aorta, wherein the total aortic diameter is a sum of the true lumen diameter and the false lumen diameter. As shown, the false lumen 28 may form along the inner curve 42 (FIG. 1), the greater outer curve 40 (FIG. 1), along both inner and outer curves 42, 40, or can be circumferential about the aorta. The false lumen 28 defines a false lumen diameter 32, which may be larger than the true lumen diameter 21, and the total aortic diameter is larger than the true lumen diameter 21 alone.

Aortic dissections are commonly classified by the Stanford and DeBakey classifications. The DeBakey Classification bases classification on the location of the primary intimal tear and distal extent of dissection. For example, under DeBakey Classification, FIG. 2A illustrates a Type I aortic dissection (e.g., the dissection extends along both the ascending aorta 12 and descending aorta 14), FIG. 2B illustrates a Type II aortic dissection (e.g., the dissection is primarily along the ascending aorta 12), and FIG. 2C illustrates a Type III aortic dissection (e.g., the dissection is primarily along the descending aorta 14). The Stanford Classification is based on the location of the primary intimal tear of the dissection and does not necessarily consider the distal extent of dissection. For example, under Stanford Classification, the aortic dissections of FIGS. 2A-2B are classified as Type A aortic dissections (e.g., the primary intimal tear is along the ascending aorta 12, and optionally along the descending aorta 14) and the aortic dissection of FIG. 2C is classified as a Type B aortic dissection (e.g., the dissection is along the descending aorta 14). FIG. 2D illustrates another representation of the relative positioning of a Type A aortic dissection vs. a Type B aortic dissection under the Stanford Classification.

In Type A aortic dissections, as shown in FIGS. 2A-2B, the defect 26 is positioned along at least a portion of the ascending aorta 12. In some instances, the diseased portion 22, or defect 26, may be isolated (e.g., as shown in FIGS. 5A-5B) and in other instances may extend over a substantial length of the ascending aorta 12 (e.g., as shown in FIGS. 6A-7B). In some instances, the defect extending along the length of the ascending aorta 12, through the aortic arch, and into the descending aorta 14. In Type B aortic dissections, as shown in FIG. 2C, the diseased portion 22 is positioned along at least a portion of the descending aorta 14, and does not extend along the ascending aorta. Although subsequent device and implantation methods are shown and described with respect to the ascending aorta 12, similar treatment methods may be implemented within the descending aorta 14.

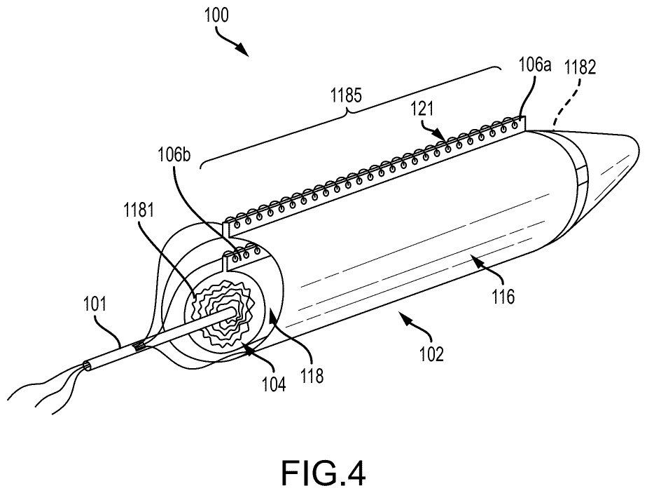

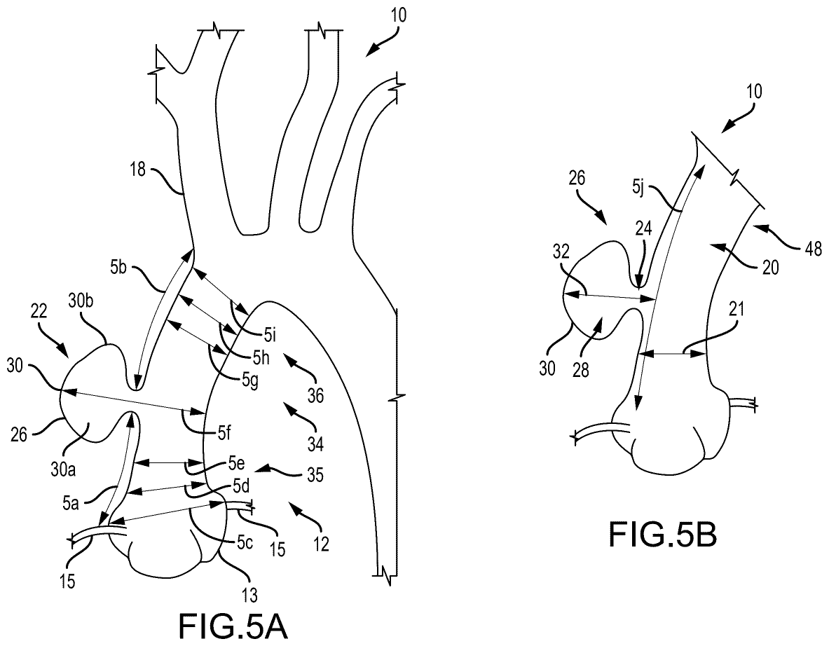

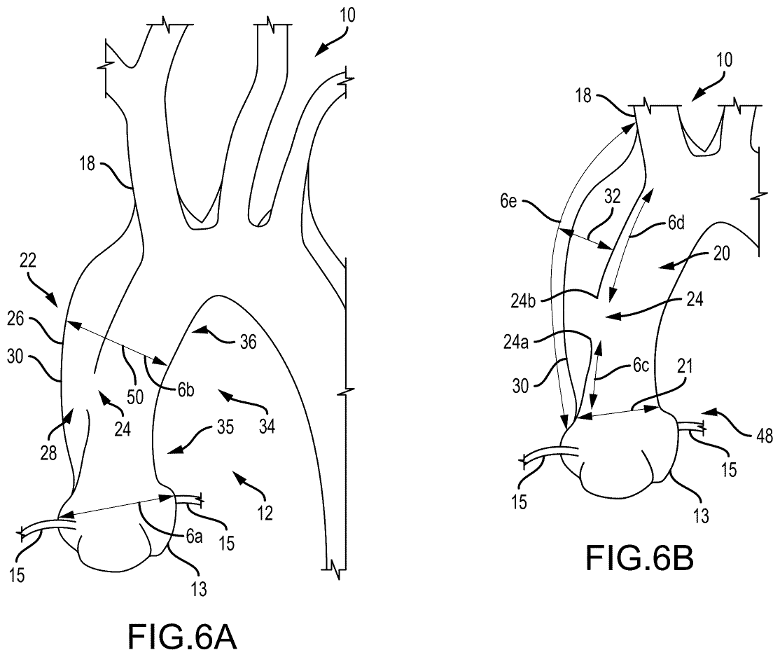

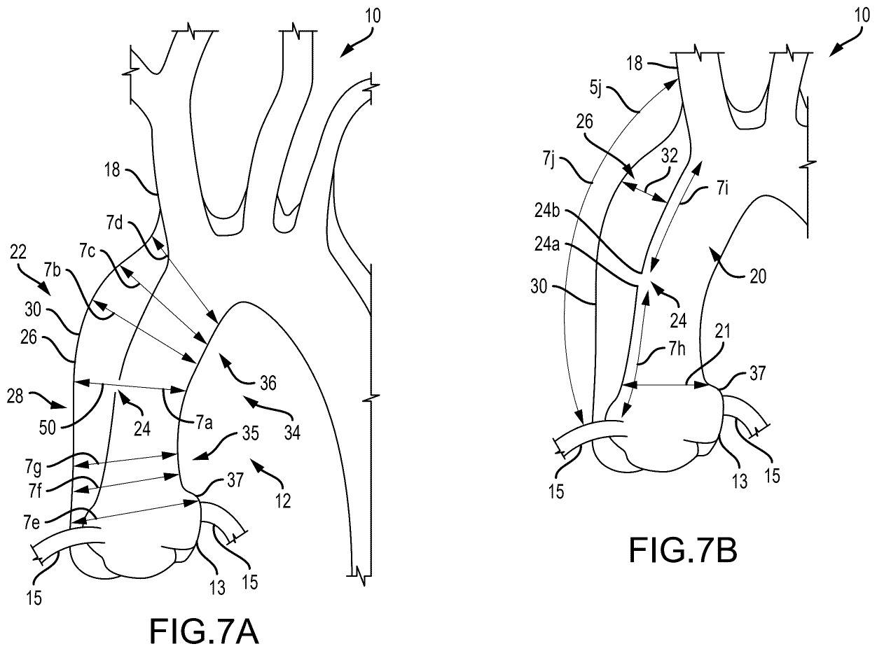

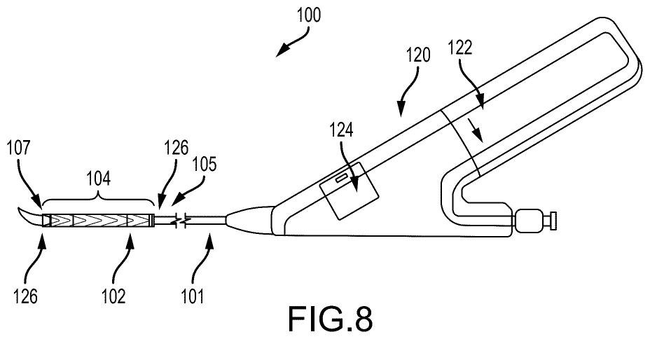

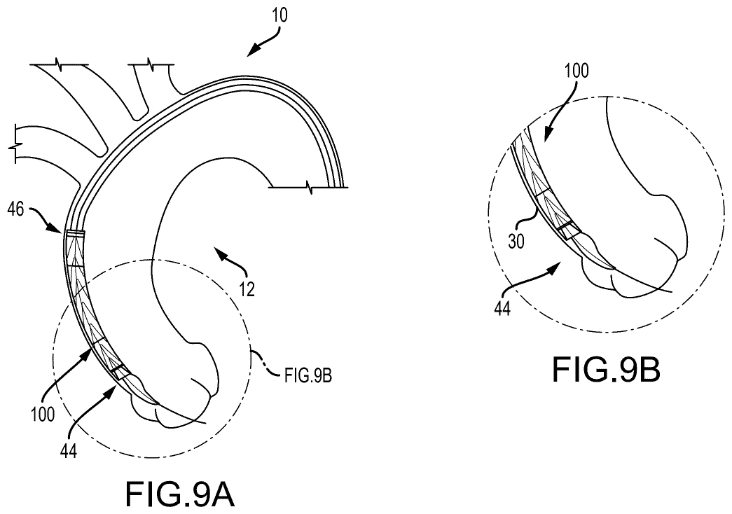

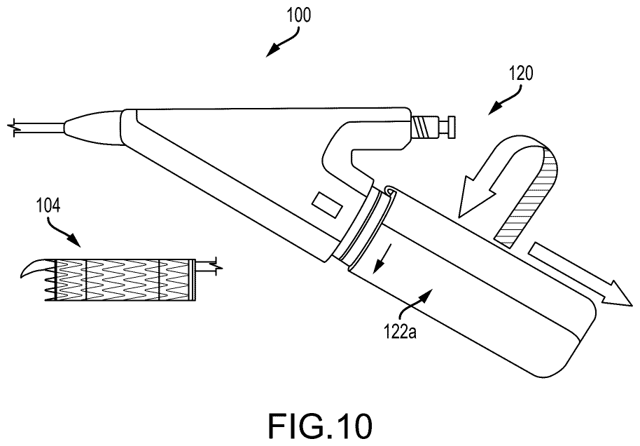

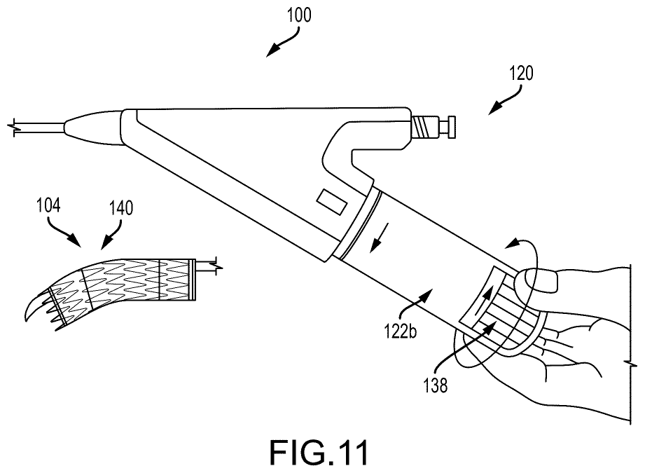

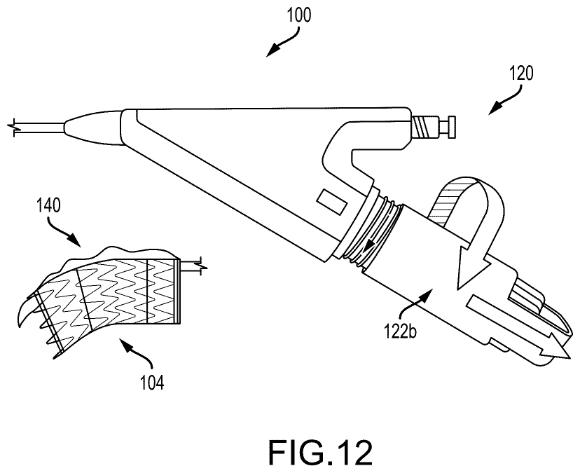

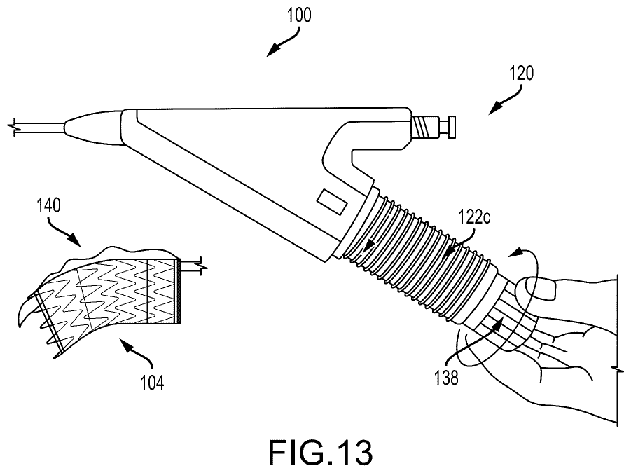

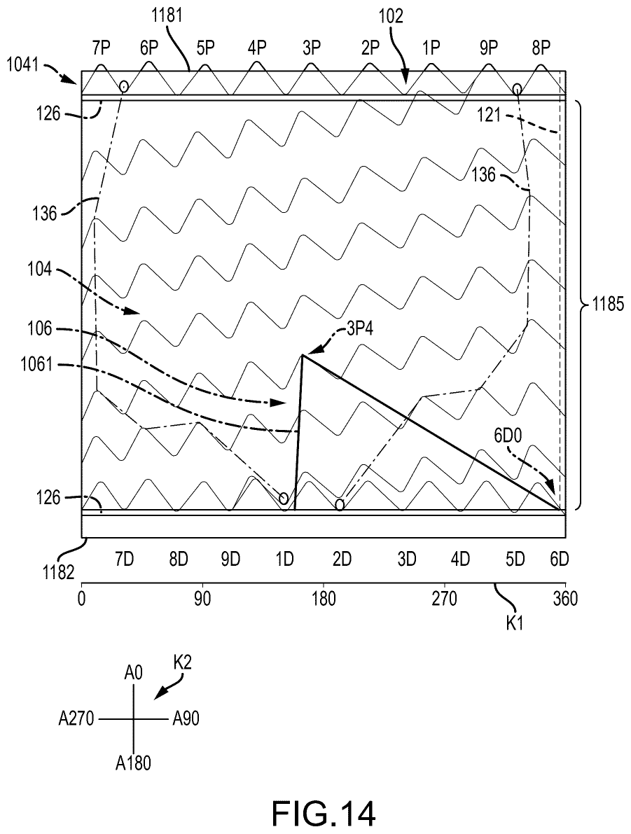

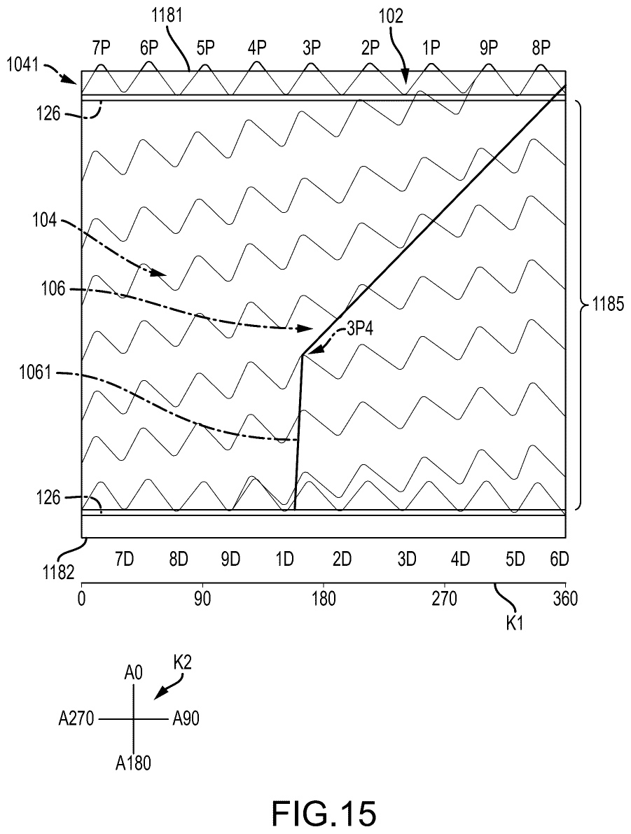

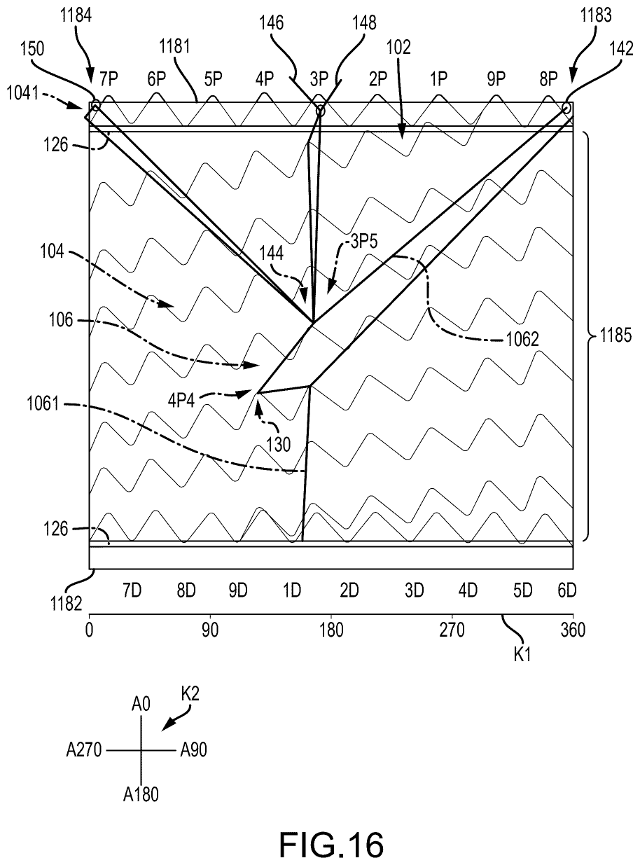

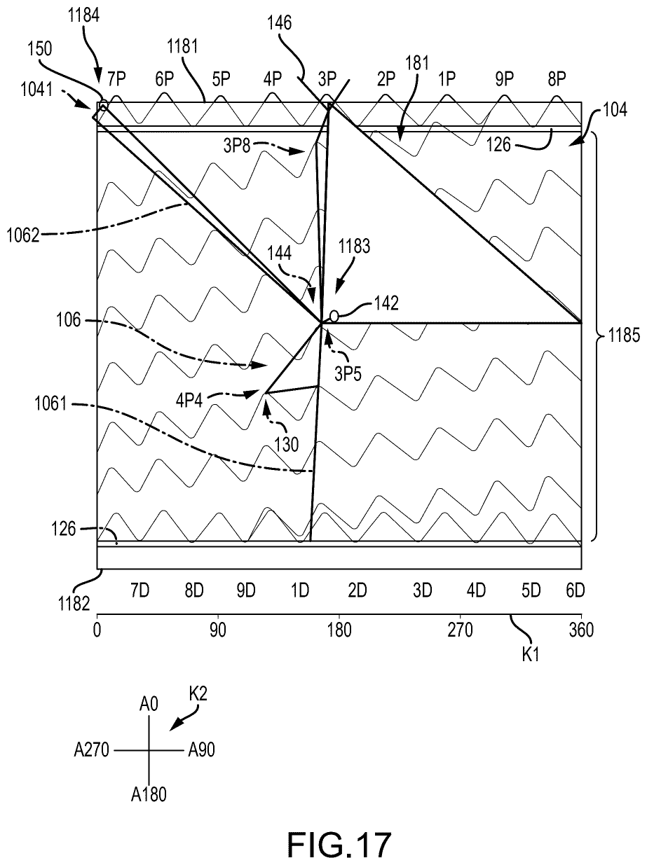

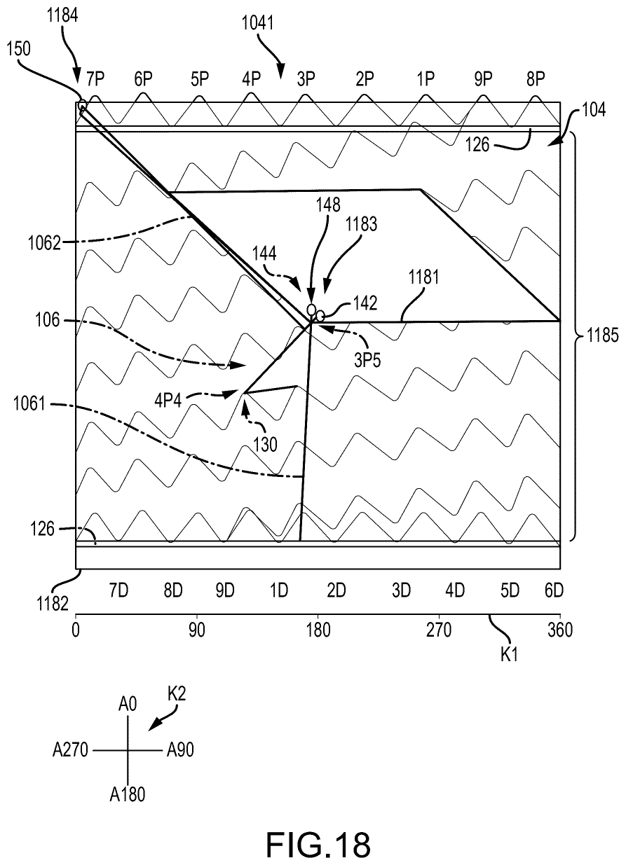

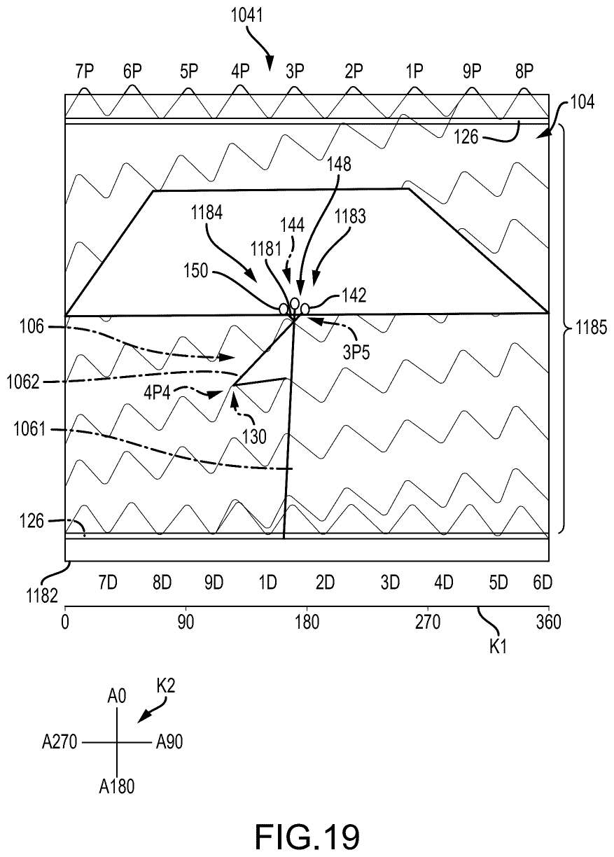

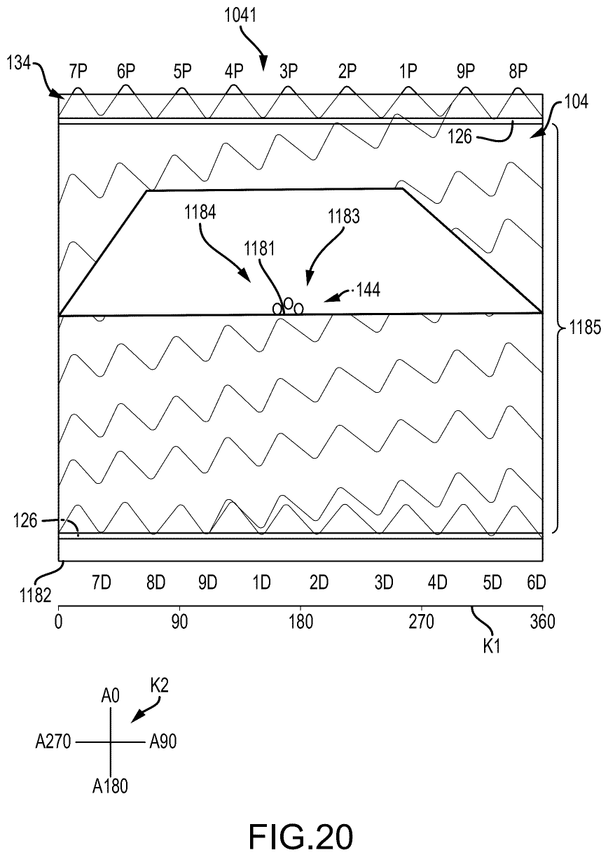

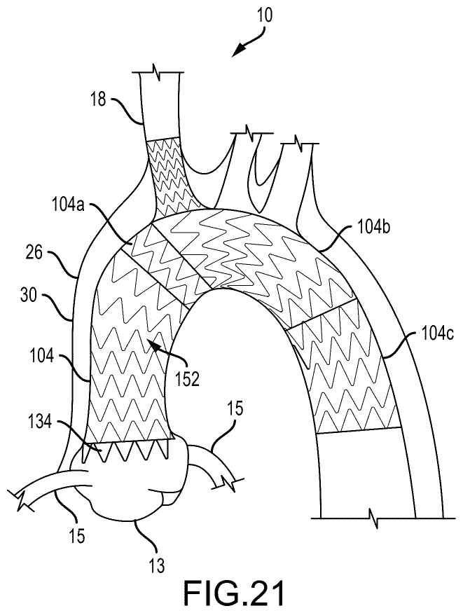

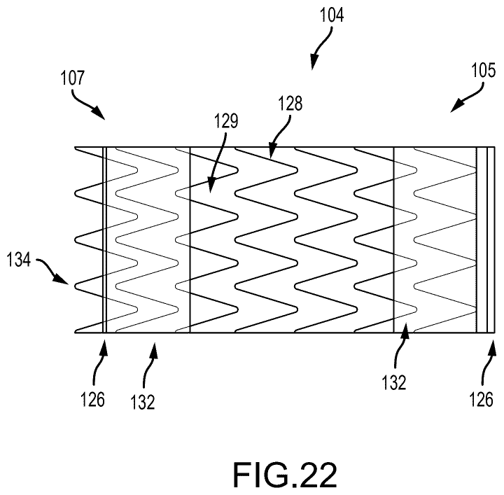

Turning to FIG. 3, in some embodiments, a delivery system 100, including a catheter 101, a constraint 102, and an endoprosthesis 104 may be delivered to the aorta 10 at the location of the diseased portion 22. The endoprosthesis 104 may be delivered within the aorta 10 when in the endoprosthesis 104 is in a collapsed state with a collapsed diameter. As shown in FIG. 3, the constraint 102 is configured to maintain the endoprosthesis 104 in the collapsed state. The constraint 102 includes a first constraint 116 and a second constraint 118 (FIG. 4), though more than two constraints are contemplated. In some embodiments, the first and/or second constraint 116, 118 include sleeves. The constraint 102 includes a body 108 maintained in a tubular shape, where the body 108 includes opposing edges 110, 112 releasably secured together with at least one fiber, or deployment line 106 (FIG. 3).

At a desired time, the constraint 102 is able to be released by releasing the opposing edges 110, 112 of the constraint 102 using the deployment line 106. The constraint 102 is optionally secured to the endoprosthesis 104 such that the constraint 102 remains in the body of the patient following release of the constraint 102; otherwise, the constraint 102 or at least a portion of the constraint 102 may be removed after deployed. For example, a portion of the body 108 of the constraint 102 may be secured to the endoprosthesis 104 (e.g., using a suture or fiber) such that the constraint 102 stays in the patient's body with the endoprosthesis 104 post-deployment.

As shown, the constraint 102 is arranged along a length of the endoprosthesis 104 and circumferentially around the endoprosthesis 104, so that at least a portion of (e.g., some or all of the length of) the endoprosthesis 104 is covered and/or constrained for delivery. The deployment line 106 may be arranged within a lumen (not shown) of the catheter 101 and extend toward a proximal end of the catheter 101 that is arranged external to a patient during delivery of the endoprosthesis 104. The deployment line 106 includes a proximally-extending portion 114, or end 114 that a user may apply tension to in order to release the constraint 102 and deploy the endoprosthesis 104. The endoprosthesis 104 may be a stent, stent-graft, a balloon, filter, heart valve, or a similar device as desired.

Although other configurations of the constraint 102 can be used, a preferred configuration is a generally rectangular one having a constant width, although tapered or stepped configurations, for example, are contemplated. Apertures 142 (FIG. 16), also described as openings or eyelets, are optionally disposed along the side margins so that a coupling member, such as the deployment line 106, may be laced or threaded therethrough. The apertures 142 may be in the form of through holes, which may be formed by a uniform-diameter puncturing device or by other means such as laser-drilling. Alternatively, the apertures 142 may be formed by loops of material (e.g., fibers) which may be attached to the constraint 102 or formed by other means.

The endoprosthesis 104 has a collapsed diameter and a deployed diameter that is larger than the collapsed diameter. The removable constraint 102 is attached to the endoprosthesis 104 at its collapsed diameter. As mentioned above, the removable sleeve illustratively includes a deployment line 106 configured to release the constraint 102 and transition the endoprosthesis 104 from the collapsed diameter to the deployed diameter in response to a force applied to the deployment line 106. The endoprosthesis 104 further includes an upstream, proximal edge 110 and a downstream, distal edge 112 corresponding to upstream and downstream edges of the removable constraint 102, further discussed herein.

Referring now to FIG. 4, in some embodiments, the endoprosthesis 104 includes both a first constraint 116, and a second constraint 118. Utilization of multiple, overlapping (either partially or fully overlapping) constraints (i.e., more than two) may allow the device to be deployable between multiple (i.e., more than two) diameters. For example, the endoprosthesis 104 may be deployable from a collapsed diameter to an intermediate deployment diameter, and then to a final deployment diameter. In some instances, the second constraint 118 is configured to constrain the endoprosthesis 104 at an intermediate diameter during delivery, while the first constraint 116 is configured to be positioned about the second constraint 118 to constrain the device to the collapsed diameter prior to delivery. In such an embodiment, the first constraint 116 must be deployed at the same time as or before the second constraint 118 may be deployed. The first constraint 116 may include a first deployment line section 106a, which allows a user to deploy the first constraint 116 independently from the second constraint 118. For example, actuation of the first deployment line section 106a results in release of the device from the collapsed diameter to an intermediate diameter of the device. Similarly, the second constraint 118 may include a second deployment line section 106b. For example, actuation of the second deployment line section 106b results in release of the endoprosthesis 104 to an expanded diameter greater than the intermediate diameter. In some embodiments, the deployment line sections 106a, 106b comprise at least two separate lines, which are actuated independently in order to effect deployment of the respective first constraint 116 and second constraint 118. In other embodiments, the deployment line sections 106a and 106b comprise a single deployment line 106, which may be actuated to deploy the first constraint 116, and then further actuated to deploy the second constraint 118. In either embodiment, the second constraint 118 may be deployed immediately upon deployment of the first constraint 116, or a time after deployment of the first constraint 116. For example, after deployment of the first constraint 116, a user may immediately deploy the second constraint 118. Otherwise, a discretionary period of time may pass between the deployment of the first constraint 116 and deployment of the second constraint 118. In some embodiments, and as described further below, a user may reposition or otherwise adjust a position of the endoprosthesis 104 within the aorta 10 between deployment of the first constraint 116 and the second constraint 118.

Turning to FIGS. 5A-7B, prior to delivering and implanting the endoprosthesis 104 within the aorta 10, the ascending aorta 12 of the patient may be assessed. Assessment of the ascending aorta 12 may assist in determining if a patient meets the anatomical requirements for implantation and/or is compatible with the implantation procedure. Assessment of the ascending aorta 12 including taking anatomical measurements 34 at multiple locations along a length of the ascending aorta 12. The anatomical measurements 34 include length and diameter measurements, as subsequently described. The anatomical measurements 34 may be taken by a medical professional utilizing a contrast-enhanced computed tomographic angiography (CTA) or other imaging techniques. Assessment of the ascending aorta 12 may also help determine sizing of the endoprosthesis 104, and in particular, the deployed diameter of the endoprosthesis 104.

In one example, as shown in FIGS. 5A-5B, the defect 26 may be an isolated defect positioned along only a portion of a length of the ascending aorta 12. The defect 26 defines a false lumen 28 and a the false lumen wall 30 extending outwardly from the true lumen 21. A first measurement 5a may be taken between the distal-most coronary artery 15 and a proximal extent 30a of the primary intimal tear, which is optionally at a proximal extent of the false lumen wall 30 of defect 26. The first measurement 5a should be about 2 cm or larger to ensure a sufficient sized proximal landing zone 44 (FIG. 9A) for the endoprosthesis 104 during deployment within the aorta 10. A second measurement 5b may be taken between a distal extent 30b of the primary intimal tear, which is optionally at a distal extent of the false lumen wall 30 of defect 26 and the brachiocephalic artery 18. The second measurement 5b should be at least about 2 cm or larger to ensure a sufficient sized distal landing zone 46 (FIG. 9A) for the endoprosthesis 104 during deployment within the aorta 10.

A third measurement 5c may be measured at a dimeter of a proximal aortic neck 35, which may be substantially the same as the true lumen diameter 21, or depending on the location of the defect 26, may be substantially the same as the total aorta diameter 5f. Optionally, a fourth measurement 5d and a fifth measurement 5e may be taken at the diameter of the proximal aortic neck 35, where the fourth and fifth measurements 5d, 5e are about 1 cm apart from each other and the fourth measurement 5d is about 1 cm apart from the third measurement 5c. In other embodiments, more than three measurements or fewer than three measurements may be taken at the diameter of the proximal aortic neck 35. The third, fourth, and fifth measurements 5c-5e are measured in a non-diseased portion 48 of the aorta 10, or in other words, a region excluding the defect 26. The diameter of the proximal aortic neck 35, which depending on the location of the defect 26 may be substantially the same as the total aorta diameter 5f, may be between about 27 mm and 48 mm, optionally about 27 mm, optionally between about 27 mm and 30 mm, optionally between about 30 mm and 35 mm, optionally between about 35 mm and 40 mm, optionally between about 40 mm and 45 mm, optionally between about 45 mm and 48 mm, or optionally 48 mm. The diameter of the proximal aortic neck 35 may indicate a minimum deployed diameter for the endoprosthesis 104.

A sixth measurement 5f may be taken at a maximum defect diameter, which extends from the false lumen wall 30 to the inner wall 17 of the true lumen (FIG. 1) of the aorta 10 (e.g., across the greater outer curve 40 and the inner curve 42). The maximum defect diameter includes the largest total diameter of the true lumen 21 and false lumen 32. The sixth measurement 5f defines a maximum transverse aortic diameter 50, which is the maximum sum of the false lumen diameter 32 and the true lumen diameter 21. In some embodiments, and as discussed further herein, the deployed diameter of the endoprosthesis 104 may be tuned such that the deployed diameter is at least as large as the maximum transverse aortic diameter 50. In some embodiments, the diameter of the endoprosthesis 104 is about 31 mm or greater, optionally about 34 mm, optionally about 37 mm, optionally about 40 mm, optionally about 45 mm, optionally about 49 mm, optionally about 53 mm, or optionally greater than 53 mm. The diameter of the endoprosthesis 104 may be oversized compared to the diameter measurements.

A seventh measurement 5g may be taken at a distal aortic neck 36, which may be substantially the same as the true lumen diameter 21. Optionally, an eighth measurement 5h and a ninth measurement 5i may be taken at the diameter of the distal aortic neck 36, where the eighth and ninth measurements 5h, 5i are about 1 cm apart from each other and the eighth measurement 5h is about 1 cm apart from the seventh measurement 5g. In other embodiments, more than three measurements or fewer than three measurements may be taken at the distal aortic neck 36. The seventh, eighth, and ninth measurements 5g-5i are measured in the non-diseased portion 48 of the aorta 10. The diameter of the distal aortic neck 36 may be between about 27 mm and 48 mm, optionally about 27 mm, optionally between about 27 mm and 30 mm, optionally between about 30 mm and 35 mm, optionally between about 35 mm and 40 mm, optionally between about 40 mm and 45 mm, optionally between about 45 mm and 48 mm, or optionally 48 mm. The diameter of the distal aortic neck 36 may indicate a minimum deployed diameter for the endoprosthesis 104.

A tenth measurement 5j may be taken along a total treatment length of the ascending aorta 12. The tenth measurement 5j may be taken along the greater curve 40, and includes a length of the defect 26.

In another example, turning to FIGS. 6A-6B, in some embodiments, the defect 26 extends along a substantial length of the ascending aorta 12. Anatomical measurements 34 may be taken in any order to assess the aorta 10 of the patient, similar to the procedure discussed with respect to FIGS. 5A-5B. As shown, the tear region 24 of the diseased portion 22 extends along a portion of the length of the ascending aorta 12 and is positioned between the proximal and distal aortic necks 35, 36.

A first measurement 6a may be taken at a diameter of the proximal aortic neck 35. The first measurement 6a is taken at a proximal extent of a proximal landing zone 44 (FIG. 9A), which can be in the diseased portion 22 of the aorta 10 or in the non-diseased portion 48 of the aorta 10 (e.g., not within a region adjacent to the defect 26). In some embodiments, the first measurement 6a is taken at a position adjacent the coronary arteries 15. The first measurement 6a may be substantially the same as the true lumen diameter 21 and may indicate a minimum deployed diameter for the endoprosthesis 104. In embodiments, the diameter of the endoprosthesis 104 is oversized relative to the true lumen diameter 21. Optionally, more than one measurement may be taken at the diameter of the proximal aortic neck 35. The diameter of the proximal aortic neck 35 may be between about 27 mm and 48 mm, optionally about 27 mm, optionally between about 27 mm and 30 mm, optionally between about 30 mm and 35 mm, optionally between about 35 mm and 40 mm, optionally between about 40 mm and 45 mm, optionally between about 45 mm and 48 mm, or optionally 48 mm.

A second measurement 6b may be taken at the maximum transverse aortic diameter 50, which is the maximum sum of the false lumen diameter 32 and the true lumen diameter 21. More than one measurement may be taken to determine the location of the maximum transverse aortic diameter 50. The maximum transverse aortic diameter 50 may be positioned anywhere along the diameter of the diseased portion 22. In some embodiments, the false lumen diameter 32 is about 30 mm or greater, optionally about 34 mm, optionally about 37 mm, optionally about 40 mm, optionally about 45 mm, optionally about 49 mm, optionally about 53 mm, or optionally greater than 53 mm. The endoprosthesis 104 deployed diameter may be tuned such that the deployed diameter is at least as large as the maximum transverse aortic diameter 50.

A third measurement 6c may be taken at a distance between the distal most coronary artery 15 and a proximal end 24a of the tear region 24, wherein the tear region 24 may be at the primary intimal tear. The third measurement 6c should be about 2 cm or greater to ensure a properly sized proximal landing zone 44 (FIG. 9A) for the endoprosthesis 104 at deployment.

A fourth measurement 6d may be taken at a distance between a distal end 24b of the tear region 24 to the brachiocephalic artery 18. The fourth measurement 6d should be about 2 cm or greater to ensure a properly sized distal landing zone 46 (FIG. 9A) for the endoprosthesis 104 at deployment.

A fifth measurement 6e may be taken at a total treatment length from the distal-most coronary artery 15 and the brachiocephalic artery 18. The fifth measurement 6e may be taken along the outer curve of the ascending aorta and along the false lumen wall 30. The fifth measurement 6e may determine a maximum longitudinal length of the endoprosthesis 104. In some embodiments, the longitudinal length of the endoprosthesis is about 7 cm or greater, optionally about 8 cm, optionally about 9 cm, or optionally about 10 cm. In situations where the fifth measurement 6e is larger than about 10 cm, more than one endoprosthesis 104 may be deployed to substantially cover the length of the ascending aorta 12. The use of more than one endoprosthesis 104 is discussed below with respect to FIG. 21.

In a further example, turning to FIGS. 7A-7B, in some embodiments, the defect 26 extends along a substantial length of the ascending aorta 12, including at the root 13. Anatomical measurements 34 may be taken in any order to assess the aorta 10 of the patient, similar to the procedure discussed with respect to FIGS. 5A-6B.

In some previous scenarios, a stent graft, for example a GORE® TAG® Conformable Thoracic Stent Graft, may have been used for treating Type B dissections, where in some embodiments it is taught to have greater than or equal to about 20 mm proximal landing zone 44 proximal to the primary entry tear 24a of the tear region 24, and where the proximal extent of the landing zone must not be dissected. The sizing for Type B dissection is based on the single aortic diameter (e.g., in the true lumen 20) at the most proximal extent of the proximal landing zone 44 where the aorta 10 is not dissected. The greater than or equal to about 20 mm proximal landing zone can include both dissected and non-dissected aorta. As a comparison, in this disclosure, a stent graft, e.g., a GORE® Ascending Stent Graft, may be used in treatment of Acute Type A dissections and in some embodiments may have different sizing requirements (as compared to a GORE® TAG® Conformable Thoracic Stent Graft, for example). For example, in some embodiments, there may be at least 20 mm from the distal coronary artery 15 and the primary entry tear 24a, and the dissection can extend into the aortic root 13.

Therefore, the landing zone 44 can include dissected (e.g., the diseased portion 22) and non-dissected aorta (e.g., the non-diseased portion 48), or can be all dissected. Device sizing may be selected based on total aortic diameters (including true lumen 20 and false lumen 28) within the proximal landing zone 44.

Similar to FIGS. 6A-6B, a first measurement 7a may be taken at the maximum transverse aortic diameter 50, which is the maximum sum of the false lumen diameter 32 and the true lumen diameter 21 (FIG. 1). More than one measurement may be taken to determine the location of the maximum transverse aortic diameter 50. The maximum transverse aortic diameter 50 may be positioned anywhere along the diameter of the diseased portion 22. In some embodiments, the false lumen diameter 32 is about 30 mm or greater, optionally about 34 mm, optionally about 37 mm, optionally about 40 mm, optionally about 45 mm, optionally about 49 mm, optionally about 53 mm, or optionally greater than 53 mm. The endoprosthesis 104 deployed diameter may be tuned such that the deployed diameter is at least as large as the maximum transverse aortic diameter 50.

A second measurement 7b may be measured at a diameter of a distal aortic neck 36, which may be substantially the same as the true lumen diameter 21. Optionally, a third measurement 7c and a fourth measurement 7d may be taken at the diameter of the distal aortic neck 36, where the third and fourth measurements 7c, 7d are about 1 cm apart from each other and the third measurement 7c is about 1 cm apart from the second measurement 7b. The diameter of the distal aortic neck 36 may be between about 27 mm and 48 mm or larger than 48 mm, optionally about 27 mm, optionally between about 27 mm and 30 mm, optionally between about 30 mm and 35 mm, optionally between about 35 mm and 40 mm, optionally between about 40 mm and 45 mm, optionally between about 45 mm and 48 mm, optionally 48 mm, or optionally larger than 48 mm. The average diameter of the distal aortic neck 36 may indicate a minimum deployed diameter for the endoprosthesis 104.

Optionally, a fifth measurement 7e may be measured at a diameter of the proximal aortic neck 35, which may be substantially the same as the true lumen diameter 21. Optionally, a sixth measurement 7f and a seventh measurement 7g may be taken at the diameter of the proximal aortic neck 35, where the measurements are about 1 cm apart from each other. The average diameter of the proximal aortic neck 35 may be between about 27 mm and 48 mm, optionally about 27 mm, optionally between about 27 mm and 30 mm, optionally between about 30 mm and 35 mm, optionally between about 35 mm and 40 mm, optionally between about 40 mm and 45 mm, optionally between about 45 mm and 48 mm, or optionally 48 mm. The average diameter of the proximal aortic neck 35 may indicate a minimum deployed diameter for the endoprosthesis 104.

An eighth measurement 7h may be taken at a distance between the distal most coronary artery 15 and a proximal end 24a of the tear region 24, wherein the tear region 24 may be at the primary intimal tear, which should be about 2 cm or greater to ensure a properly sized proximal landing zone 44 (FIG. 9A) for the endoprosthesis 104 at deployment.

A ninth measurement 7i may be taken at a distance between a distal end 24b of the tear region 24 to the brachiocephalic artery 18, which should be about 2 cm or greater to ensure a properly sized distal landing zone 46 (FIG. 9A) for the endoprosthesis 104 at deployment.

A tenth measurement 7j may be taken at a total treatment length from the distal-most coronary artery 15 and the brachiocephalic artery 18. The tenth measurement 7j may be taken along the outer curve of the ascending aorta 12. The tenth measurement 7j may determine a maximum longitudinal length of the endoprosthesis 104. As discussed above, more than one endoprosthesis 104 may be deployed to substantially cover the length of the ascending aorta 12.

In addition to the anatomical measurements 34 as discussed with respect to FIGS. 5A-7B, the aorta 10 may be visually assessed by the medical professional. For example, the proximal and distal landing zones 44, 46 (FIG. 9A) should be free of heavy calcification and/or heavy thrombosis in the true lumen 21 to help ensure proper positioning and deployment of the endoprosthesis 104 within the aorta 10. This also helps to ensure adequate sealing between the endoprosthesis 104 and the aortic walls of the aorta 10. The endoprosthesis 104 is deployed within the true lumen 21 to facilitate the fluid bypass at the false lumen 28.

The anatomical measurements 34 discussed above may be used to select a sizing of the endoprosthesis 104, including a size of the deployed diameter of the endoprosthesis 104. For example, the anatomical measurements 34 give the user information regarding the total aorta diameter 50, which includes the false lumen diameter 32, which may be used to select the deployed diameter of the endoprosthesis 104. In some embodiments, the deployed diameter of the endoprosthesis is selected based on diametrical measurements taken at least one of the proximal aortic neck 35, the distal aortic neck 36, and the maximum transverse aortic diameter 50. For example, the deployed diameter of the endoprosthesis 104 may be at least as large as an expected diameter of the false lumen 28, where, if not measured directly, the expected diameter of the false lumen 28 can be determined by the total aorta diameter 50 minus the true lumen 21. The expected diameter of the false lumen 28 may be determined by a difference between the maximum transverse aortic diameter 50 and the true lumen diameter 21 (e.g., one or both of diametrical measurements taken at the distal aortic 36 or proximal aortic neck 35). In some embodiments, a maximum deployed diameter of the endoprosthesis 104 may be about the same as the measured maximum transverse aortic diameter 50 of the aorta 10. Similarly, a minimum deployed diameter of the endoprosthesis 104 may be selected to be about the same as the diametrical measurement at the proximal aortic neck 35, the diametrical measurement at the distal aortic neck 36, or an average thereof. In other embodiments, the deployed diameter of the endoprosthesis 104 may be selected based on an average or median of two or more of the diametrical measurements at the proximal aortic neck 35, diametrical measurements at the distal aortic neck 36, and the maximum transverse aortic diameter 50.

In some embodiments, the deployed diameter of the endoprosthesis 104 is oversized relative to one or more of the expected diameter of the false lumen 28 or the maximum transverse aortic diameter 50 to ensure the endoprosthesis 104 will contact the walls 17 of the aorta and the false lumen wall 30 upon deployment. The deployed diameter may be about 6% to 33% larger than the expected diameter of the false lumen 32, optionally from about 20% to 30% larger than the expected value of the false lumen diameter 32.

The anatomical measurements 34 discussed above may also be used to select a longitudinal length of the endoprosthesis 104. The length measurements along the greater curve 38 of the aorta 10 (e.g., a total length of the diseased portion 22 of the ascending aorta 10) may be about the same as a maximum longitudinal length of the endoprosthesis 104. The length measurements proximate to the tear region 24 (e.g., measurements 6c, 6d, 7h, and 7i) may be used to determine a minimum longitudinal length of the endoprosthesis 104. As discussed with respect to FIG. 21, more than one endoprosthesis 104 may be deployed to cover the length of the diseased portion 22 of the aorta 10. For example, a length of a first endoprosthesis 104a may be about the same as the length proximal to the tear region 24 and a length of a second endoprosthesis 104b may be about the same as the length distal to the tear region 24. In embodiments where multiple endoprostheses 104 are used, neighboring endoprostheses 104 overlap one another to allow for sufficient overlap for protection against endoleaks. In some embodiments, and depending on the diameters of the endoprostheses 104, the overlap is between about 3 cm to about 5 cm.

Turning to FIG. 8, after assessing the aorta 10, the endoprosthesis 104 may be implanted within the aorta 10. As discussed with respect to FIGS. 3-4, the delivery system 100 may be used to constrain the endoprosthesis 104 to the collapsed diameter for delivery. The delivery system 100 may include a housing 120 from which the catheter 101 extends from. The housing 120 stays external to the patient and includes a handle 122 for a user to hold during deployment of the endoprosthesis 104. The housing 120 may also include an access hatch 124 coupled to the housing 120 and allows a user to directly access the deployment line 106.

Optionally, the endoprosthesis 104 and/or the constraint 102 may include one or more radiopaque markers 126. The radiopaque markers 126 may be positioned on distal and proximal ends 105, 107 of the endoprosthesis 104 to aid in visualization of the endoprosthesis 104 and/or constraint 102 during deployment.

Turning to FIGS. 9A-9B, the endoprosthesis 104 may be delivered within the ascending aorta 12. The endoprosthesis 104 is delivered such that the endoprosthesis 104 is delivered within the true lumen 21 and substantially bypasses blood flow to the false lumen 21. The endoprosthesis 104 can extend along a substantially length of the false lumen 32 and along the greater outer curve 40 of the aorta 10. The endoprosthesis 104 extends from the proximal landing zone 44 (FIG. 9A) to the distal landing zone 46 (FIG. 9A). During delivery, the endoprosthesis 104 may be steered along the curvature of the inner curve 42 via a steering line 136 extending within the catheter 101 (not shown) and operably coupled to the endoprosthesis 104. Applying tension to the steering line (e.g., by way of the housing 120 and/or catheter 101) facilitates angulation of the endoprosthesis 104 during delivery thereof.

Turning to FIG. 10, once positioned within the ascending aorta 12, the endoprosthesis 104 may be deployed to an intermediate diameter between the collapsed diameter and the deployed diameter. Deploying to the intermediate diameter includes releasing the first constraint 116 using the deployment line 106.

The secondary constraint 118 maintains the endoprosthesis 104 in the intermediate diameter. In some embodiments, turning the handle 122 of the housing 120 disengages the handle 122 from the deployment system 100. Pulling the handle 122 and deployment line 106 away from the delivery system 100 facilitates the release of the first constraint 116. Details of the release of the first constraint 116 are discussed below with respect to FIGS. 14-20. As shown, a first portion 122a of the handle 122 (e.g., an outer portion of the handle) may be removed after turning the handle 122. When the outer, first portion 122a is removed, a second portion 122b (FIG. 12) of the handle 122 remains coupled to the housing 120.

Turning to FIG. 11, optionally after deployment to the intermediate diameter, a position of the endoprosthesis 104 may be adjusted within the aorta 10 and along the inner curve 42 by manipulation of the steering line 136 (FIG. 14) and/or by moving the catheter 101. To adjust position, the user may turn an angulation control dial 138 within the housing 120. The angulation control dial 138 is operably coupled to the steering line 136 and turning the angulation control dial 138 applies tension to the steering line 136 which causes the endoprosthesis 104 to adjust in position. In some embodiments, the endoprosthesis 104 is optionally adjusted to form a bend 140, which may substantially match a geometry of the aorta 10.

Turning to FIG. 12, after deployment to the intermediate diameter and optional repositioning of the endoprosthesis 104, the endoprosthesis 104 may be deployed to the deployed diameter. To fully deploy the endoprosthesis 104, the second constraint 118 is released using the deployment line 106. In some embodiments, turning the handle 122 of the housing 120 applies tension to the deployment line 106 to facilitate the release of the second constraint 118. As shown, the second portion 122b of the handle 122 may be removed after turning the handle 122. When the second portion 122b is removed, a third portion 122c (FIG. 13) of the handle 122 remains coupled to the housing 120. Details of the release of the second constraint 118 are discussed below with respect to FIGS. 14-20.

Turning to FIG. 13, optionally after deployment to the deployed diameter, the position of the endoprosthesis 104 may be further adjusted within the aorta 10. To adjust the position, the user may further turn the angulation control dial 138 within the housing 120 to apply tension to the steering line 136 which causes the endoprosthesis 104 to further adjust in position.

Turning to FIGS. 14-20, a release mechanism of the constraint 102 is discussed. FIGS. 14-20 illustrate the constraint 102 as a plan view in a generally flat orientation for visualization purposes. However, in practice, the constraint 102 is arranged in a tubular configuration about the endoprosthesis 104. As such, a first key “K1” is provided with each of the plan views of the constraint 102 to illustrate the degree region of the constraint 102 through which the deployment line 106 (FIG. 3) is routed. K1 is arranged linearly along a longitudinal length of the constraint 102 and the numbers represent a radial position of the constraint 102 in the tubular form. The numbers range from 0 degrees to 360 degrees, where the proximal edge 110 is positioned at 0 degrees and the distal edge 112 is positioned at 360 degrees. A second key “K2” is provided with each of the plan views and further illustrates the angles at which the deployment line is routed, the degrees of each angle being designated with an “A” and generally shown in a circular circumferential arrangement. The first and second keys K1 and K2 are meant to be references and it is noted that exact radial positions which may be different from device-to-device, as discussed below.

The endoprosthesis 104 may be configured as a stent graft, including a stent 128 and a graft 129 (FIG. 22). The constraint 102 includes a proximal edge 1181, a distal edge 1182, and a mid-region 1185 positioned between the proximal edge 1181 and the distal edge 1182. The proximal edge 1181 includes notations 1P, 2P, 3P, 4P, 5P, 6P, 7P, 8P, and 9P to correspond with proximal stent apices of the stent 128. For example, as described herein, a stent apex may include a 1PX notation, wherein the “1P” designates the column of apices on the proximal edge 1181 of the constraint 102 as labeled and the “X” designates the number of rows distal from the proximal edge 1181 in which the corresponding stent apex may be found. Similarly, the distal edge 1182 includes notations 1D, 2D, 3D, 4D, 5D, 6D, 7D, 8D, and 9D to correspond with distal stent apices of the stent 128 of the endoprosthesis 104. For example, as described herein, a stent apex may include a 1DX notation, wherein the “1D” designates the column of apices on the distal edge 1182 of the constraint 102 as labeled, and the “X” designates the number of rows proximal from the distal edge 1182 in which the corresponding stent apex may be found.

The notations of each stent apices may further be interchangeably referred to as “anchor points,” wherein each anchor point, or stent apex, may or may not be used to anchor the deployment line 106 as further described herein. “Stent apex” or “stent apices” may be used interchangeably with “anchor point” or “anchor points” or may further be used interchangeably with “reference point” or “reference points” wherein each reference point refers to a point on the endoprosthesis 104 corresponding with an anchor point and/or stent apex closest to the given notation. The routing pattern provided herein may be applied to a varying number of devices having different sizes. In other words, the routing pattern may be scaled to apply to any device, wherein the notations referenced herein refer to the reference point, anchor point, or stent apex located closest to the notation after scaling of the pattern.

Referring now to FIGS. 14-15, a plan view of the constraint 102 is shown. The features may be applied to one or both of the first constraint 116 or second constraint 118. As portrayed, the constraint 102 is optionally transparent so that underlying features (e.g., the endoprosthesis 104) may be viewed beneath the constraint 102. Optionally, the endoprosthesis 104 may include radiopaque markers 126 for visibility of the endoprosthesis 104 when positioned within a patient. In some instances, the endoprosthesis 104 may further include one or more steering lines 136 to facilitate bending and steering of the endoprosthesis 104 through the vasculature of a patient during delivery and/or deployment.

The deployment line 106 defines a first deployment line section 1061, and a second deployment line section 1062 (FIG. 16) As shown in FIG. 14, the first deployment line section 1061 is routed underneath the constraint 102 prior to deployment. For example, the first deployment line section 1061 may be routed between the constraint 102 and the endoprosthesis 104 from near the distal edge 1182 of the constraint 102 to a first anchor point (e.g., a first stent apex) 3P4 positioned proximally of the distal edge 1182 between the approximately 160°region and the approximately 200° region of the constraint 102 (e.g., see K1), routed under the first anchor point 3P4, and then to a starting point of a seam line 121 near a second anchor point 6D0 (e.g., a second stent apex) positioned distally at an angle of between approximately A110° and approximately A160° from the first anchor point 3P4 to begin deployment in the 360° region of the constraint 102 (e.g., see K2). As the first deployment line section 1061 is actuated, the constraint 102 (e.g., corresponding to first constraint 116 or second constraint 118) is deployed along seam line 121 in a proximal direction generally along the 360° region of the constraint 102. After deployment of the constraint 102 is complete, the first deployment line section 1061 has completed deployment and has changed position, as shown in FIG. 15. As shown, the seam line 121 is released at deployment. In embodiment, the first constraint 116 is released and remains attached to the second constraint 118 via at least one attachment point of the deployment line 106 routing. For example, the first constraint 116 may define a first aperture and the deployment line may extend through the first aperture to maintain a coupling of the two constraints 116, 118.

Upon deployment of the constraint 102, at least a portion of the constraint 102 may overhang at least a portion of the proximal edge 1041 of the endoprosthesis 104, which may in turn block or impede the flow of blood in the patient. To ensure efficient and unhindered flow of blood through the patient and the medical device, the overhanging portion of the sleeve may be pulled, peeled, retracted, bunched, pleated, everted, folded, translated, or otherwise moved back from the proximal end 107 of the endoprosthesis 104 as described further herein.

Now referring to FIG. 16, the constraint 102 includes at least a first corner region 1183. In some instances, the constraint 102 further includes a second corner region 1184. Each of the first corner region 1183 and the second corner region 1184 become further defined upon deployment. The first corner region 1183 includes an eyelet or aperture 142, through which the second deployment line section 1062 is routed. The second deployment line section 1062 is then routed under a third anchor point 3P5 (e.g., a third stent apex) positioned proximally at an angle of between approximately A200° and approximately A250° from the first corner region 1183 between the approximately 160° region and the approximately 200° region of the constraint 102, which serves as a base point 144. In other instances, multiple base points may be utilized. In any embodiment, the base point 144 may be positioned at any anchor point corresponding with the mid-region 1185 of the constraint 102 and may vary depending on the nature of the endoprosthesis 104, the diameter of the endoprosthesis 104, and other factors.

After reaching the third anchor point 3P5 from the first corner region 1183, the second deployment line section 1062 is routed under the third anchor point 3P5, and then routed up near the proximal edge 1181 of the constraint 102, for example, to an aperture 148 near anchor point 3P8, positioned proximally, and generally longitudinally, to the third anchor point 3P5. The second deployment line section 1062 then may attach to a leading attachment fiber 146. For example, in instances including the first constraint 116 and the second constraint 118, the arrangement may be utilized for either one or both of the first constraint 116 or the second constraint 118. In one example, where the constraint 102 is the second constraint 118, the leading attachment fiber 146 may be attached to both the first constraint 116 and the second constraint 118 to facilitate the attachment of the second deployment line section 1062 to the first constraint 116.

In various examples, the second deployment line section 1062 is routed through the aperture 148 located laterally between apertures 142, 150 defining a coupling point with the leading attachment fiber 146 back to the third anchor point 3P5, positioned distally, and generally longitudinally of the coupling point with the leading attachment fiber 146. The second deployment line section 1062 is again routed, or passed, under the third anchor point 3P5 and is then routed to the second corner region 1184 positioned proximally from the third anchor point 3P5 and laterally from the first corner region 1183. The second corner region 1184 includes the second aperture 150, through which the second deployment line section 1062 is routed. The second deployment line section 1062 is then routed back to the third anchor point 3P5 and routed under, passed under, or otherwise slidably anchored to the third anchor point 3P5. The second deployment line section 1062 is routed from the third anchor point 3P5 to a fourth anchor point 4P4 (e.g., a fourth stent apex) positioned distally at an angle of between approximately A200° and approximately A250° from the third anchor point 3P5 between the approximately 110° region and the approximately 160° region of the constraint 102, and is further routed under the fourth anchor point 4P4, which acts as a friction point 130 to provide friction to the deployment line 106 to facilitate the actuation of the deployment line 106. The friction point 130 helps prevent the deployment line 106 from slipping out of place, or releasing, before deployment of both the first deployment line section 1061 and the second deployment line section 1062 is complete. In an illustrative embodiment, only one friction point 130 is used, which helps prevent the endoprosthesis 104 from slipping while the deployment line 106 is actuated. However, in various instances, multiple friction points 130 may be utilized and be positioned in a variety of places on the endoprosthesis 104.

Referring now to FIGS. 17-19, as the deployment line 106 is actuated, after deployment of the first deployment line section 1061 and resultant deployment of the constraint 102, the continued actuation of the second deployment line section 1062 results in movement (e.g., pulling, retraction, bunching, pleating, eversion or folding) of the constraint 102 according to the routing pattern of the second deployment line section 1062. For example, after deployment of the first deployment line section 1061, as the second deployment line section 1062 is actuated, the first corner region 1183 is pulled, peeled, retracted, bunched, pleated, everted, folded, translated, or otherwise moved back from the proximal edge 1041 of the endoprosthesis 104 as shown in FIG. 17 to be gathered toward the base point 144.

As the second deployment line section 1062 continues to be actuated via actuation of the deployment line 106, the upstream, proximal edge 1041 of the constraint 102 is pulled, peeled, retracted, bunched, pleated, everted, folded, translated, or otherwise moved back from the proximal edge 1181 of the endoprosthesis 104 as shown in FIGS. 18-19 to be gathered at the base point 144. In an embodiment previously described having a first constraint 116 and second constraint 118, where the constraint 102 is the second constraint 118, the first constraint 116 may also be pulled, peeled, retracted, bunched, pleated, everted, folded, translated, or otherwise moved back from the proximal edge 1181 of the endoprosthesis 104 to be gathered toward the base point 144 by virtue of the attachment between the first and second constraints 116, 118.

After actuation of the upstream, proximal edge 1181 of the constraint 102, as the second deployment line section 1062 continues to be actuated via the deployment line 106, the second corner region 1184 is pulled, peeled, retracted, bunched, pleated, everted, folded, translated, or otherwise moved back from the proximal edge 1041 of the endoprosthesis 104 as shown in FIG. 19 to be gathered at the base point 144.

As shown in FIG. 20, in some instances, after the corner region(s) 1183, 1184 and the proximal edge 1181 of the constraint 102 have been moved back to the base point 144, the user may continue to apply tension to the second deployment line section 1062. As tension is continually applied, the deployment line 106 is released from the constraint 102 (and optionally any additional constraints, such as the first constraint 116), and the third anchor point 3P5 or other anchor point located at the base point 144 and is then able to be retracted or pulled out through the catheter 101 (FIG. 3). In other instances, the deployment line 106 may not be released from the constraint 102, any other sleeve, and/or the base point 144.

In some instances, after full deployment and removal of the deployment line 106, the proximal end 107 of the endoprosthesis 104 is unhindered by constraint overhang as described in detail above. As shown in FIG. 21, this in turn leaves the coronary arteries 15 substantially unobstructed by continuous blood flow therethrough. Thus, in various examples, the endoprosthesis 104 and constraint 102 are configured such that upon the constraint 102 being pulled, retracted, bunched, pleated, everted, folded or otherwise moved away from the end of the endoprosthesis 104, preferably an entirety of, but at least at the coronary region of the endoprosthesis 104 is unhindered following full deployment as described in detail above and with reference to FIGS. 17-19.

Turning to FIG. 21, at the deployed diameter, the endoprosthesis 104 may be positioned within the ascending aorta 12 such that the endoprosthesis 104 engages the false lumen wall 30 of the aorta 10 and defines a flow bypass 152 through the defect 26. In some embodiments, the endoprosthesis 104 may engage about 20 mm of the false lumen wall 30, in other embodiments, greater than about 20 mm may be observed, for example, greater than about 30 mm, greater than about 40 mm, greater than about 50 mm, or greater than about 60 mm.

In some embodiments, to cover a full length of the diseased portion 22 of the aorta 10, more than one endoprosthesis 104 may be deployed therein. In some embodiments, this occurs when the total length of the diseased portion 22 of the aorta 10 is greater than about 10 cm. For example, two or more endoprostheses 104a-104c may be implanted. In embodiments where two endoprostheses are the substantially same diameter, at least about 5 cm of overlap between the two endoprostheses should be achieved. In other embodiments where one endoprosthesis has a larger diameter than another endoprosthesis, at least about 3 cm of overlap between the two endoprostheses should be achieved. In such examples, the larger diameter endoprosthesis should be deployed into the smaller diameter endoprosthesis.

FIG. 22 shows a partial view of the endoprosthesis 104 in the deployment diameter. The endoprosthesis 104 may include the stent 128 and the graft 129 secured to the stent 128. As shown, radiopaque markers 126 may be positioned at the distal end 105 and the proximal end 107. Optionally, sealing cuffs 132 may be positioned at the distal and proximal ends 105, 107 to assist with sealing to walls 17 of the aorta 10 and the false lumen wall 30 after deployment. As shown, at the proximal end 107, a leading stent row 134 may be substantially uncovered and positioned at or proximate to the coronary arteries 15 to leave the coronary arteries 15 substantially unobstructed by the endoprosthesis 104 and the constraint to maintain blood flow to the vasculature.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A method of implanting an endoprosthesis within an aorta of a patient, the method comprising:
positioning the endoprosthesis within the aorta in a collapsed state with a constraint, the aorta having a true lumen and a false lumen due to the aorta having a defect, the true lumen defining a true lumen diameter and the false lumen formed by a false lumen wall defining a false lumen diameter, the endoprosthesis having a deployed diameter and a collapsed diameter; and
deploying the endoprosthesis to the deployed diameter such that the endoprosthesis extends along the true lumen and along at least a portion of the false lumen at a position where the defect is located in the aorta such that the endoprosthesis defines a flow bypass through a diseased portion of the aorta.

2. The method of claim 1, wherein the constraint includes a first constraint and a second constraint, and further wherein deploying the endoprosthesis includes:
releasing the first constraint constraining the endoprosthesis such that the endoprosthesis expands from the collapsed diameter to an intermediate diameter that is greater than the collapsed diameter;
repositioning the endoprosthesis; and
releasing the second constraint constraining the endoprosthesis such that the endoprosthesis expands from the intermediate diameter to the deployed diameter.

3. The method of claim 1, further including pulling back a portion of the constraint such that left and right coronary arteries are left substantially unobstructed by the endoprosthesis.

4. The method of claim 1, wherein the aorta is the ascending aorta.

5. The method of claim 1, wherein the endoprosthesis diameter is about 31 mm, 34 mm, 37 mm, 40 mm, 45 mm, 49 mm, 53 mm or more.

6. The method of claim 1, further comprising receiving information regarding a false lumen diameter and selecting the deployed diameter of the endoprosthesis to be at least as large as the true lumen diameter.

7. The method of claim 1, wherein the diseased portion of the aorta is formed by an aortic dissection.

8. The method of claim 1, wherein the deployed diameter of the endoprosthesis is selected to be from 6% to 33% or optionally from 20% to 30% larger than the total aorta diameter defined by a sum of the false lumen diameter and the true lumen diameter.

9. The method of claim 1, wherein the deployed diameter is selected based on overall diameter measurements of the aorta at a proximal aortic neck, a distal aortic neck, and a maximum transverse aortic diameter.

10. The method of claim 1, wherein the deployed diameter is selected to be from 6% to 33% larger than a diameter of the aorta proximal to a tear of the diseased portion.

11. The method of claim 1, wherein the endoprosthesis extends within the true lumen and the portion of the false lumen.

12. A method of assessing an ascending aorta of a patient, the method comprising:
measuring at least one measurement of a transverse aortic diameter that is a sum of a diameter of a true lumen and diameter of a false lumen of the ascending aorta;
selecting a deployed diameter of an endoluminal device to be compatible with any of the measured transverse aortic diameters; and
implanting the endoluminal device within the aorta.

13. The method of claim 12, further including ensuring the distance between a distal coronary artery and the proximal extent of a defect associated with the false lumen is about 2 cm or longer.

14. The method of claim 12, further including at least one of:
measuring a distance between a coronary artery and a proximal extent of a defect;
measuring a distance between a distal extent of the defect and a brachiocephalic artery;
measuring a diameter of a proximal aortic neck;
measuring a diameter of a distal aortic neck;
measuring a maximum diameter of the defect; and
measuring a total length of the ascending aorta.

15. The method of claim 12, further including selecting the endoluminal device with the deployed diameter being oversized relative to the transverse aortic diameter.

16. An implantable system configured to be implanted within an aorta of a patient, the implantable system comprising:
an implantable medical device expandable from a collapsed diameter to a deployed diameter, wherein the deployed diameter is sized relative to a total diameter of the aorta including a true lumen diameter and a false lumen diameter;
a first constraint configured to maintain the implantable medical device at the collapsed diameter; and
a second constraint configured to maintain the implantable medical device at an intermediate diameter between the collapsed diameter and the deployed diameter.

17. The implantable system of claim 16, wherein the total of the diameter of the aorta is a diameter of aorta positioned within a proximal aortic neck of the aorta.

18. The implantable system of claim 16, wherein a diameter of the implantable medical device is oversized relative to the total diameter of the aorta.

19. The implantable system of claim 16, wherein a diameter of the implantable device is

20. The implantable system of claim 16, wherein the implantable medical device includes a stent component and a graft component.
